(19) United States
(12) Patent Application Publication
(10) Pub. No.: US 2010/0317572 A1
(43) Pub. Date: Dec. 16, 2010

(54) PHARMACEUTICAL COMPOSITION COMPRISING TESOFENSINE OR ITS ANALOGUE AND AN ANTI-OBESITY COMPOUND

(75) Inventor: Jens Damsgaard Mikkelsen, Lyngby (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/809,365

(22) PCT Filed: Dec. 18, 2008
(86) PCT No.: PCT/EP2008/067853

§ 371 Date: Aug. 23, 2010

Related U.S. Application Data
(60) Provisional application No. 61/016,054, filed on Dec. 21, 2007.

(30) Foreign Application Priority Data
Dec. 20, 2007 (DK) PA 2007 01833

(51) Int. Cl. A61K 38/22; A61K 31/46; A61K 31/397; A61K 38/26; A61K 31/55; A61K 31/485; A61K 31/5375; A61K 38/28; A61P 1/04

(52) U.S. Cl. 514/5.3; 514/304; 514/210.02; 514/7.2; 514/217.01; 514/282; 514/239.5; 514/5.9; 514/5.8

(57) ABSTRACT

This invention relates to novel pharmaceutical compositions comprising a therapeutically effective combination of a compound of Formula I and an anti-obesity compound. The pharmaceutical compositions for use according to the invention are contemplated particularly useful for combating obesity or an obesity associated disease.

### TECHNICAL FIELD

This invention relates to novel pharmaceutical compositions comprising a therapeutically effective combination of a compound of Formula I and an anti-obesity compound. The pharmaceutical compositions for use according to the invention are contemplated particularly useful for combating obesity or an obesity associated disease.

### BACKGROUND ART

Within the past decades the prevalence of obesity has risen in virtually all ethnic, racial and socioeconomic populations, in both genders and in all age groups. Obesity is associated with a significantly elevated risk for type 2 diabetes, coronary heart diseases, hypertension and numerous other major illnesses and overall mortality from all causes. Therefore, weight reduction is critical for the obese patient. Thus there is impetus for creating new and alternative treatments for management of obesity.

### SUMMARY OF THE INVENTION

Investigations carried out by the inventors have lead to the conclusion that a combination of a compound of Formula I and an anti-obesity compound constitute a particularly useful combination for use in therapy associated with obesity or an obesity associated disease.

In its first aspect the invention provides a pharmaceutical composition comprising a therapeutically effective amount of a compound of Formula I, any of its stereoisomers or any mixture of its stereoisomers, or a pharmaceutically acceptable salt thereof and an anti-obesity compound or a pharmaceutically acceptable salt thereof, together with one or more adjuvants, excipients, carriers and/or diluents.

In another aspect the invention relates to the use of a combination of a compound of Formula I, any of its stereoisomers or any mixture of its stereoisomers, or a pharmaceutically acceptable salt thereof and an anti-obesity compound or a pharmaceutically acceptable salt thereof, for the manufacture of a medicament for the treatment, prevention or alleviation of obesity or an obesity associated disease of a mammal, including a human.

In a third aspect the invention provides a kit of parts comprising at least two separate unit dosage forms (A) and (B), wherein (A) comprises a compound of Formula I, any of its stereoisomers or any mixture of its stereoisomers, or a pharmaceutically acceptable salt thereof; and (B) comprises an anti-obesity compound or a pharmaceutically acceptable salt thereof; and optionally (C) instructions for the simultaneous, sequential or separate administration of the compound of (A) and the anti-obesity compound of (B) to a patient in need thereof.

In a fourth aspect the invention provides a method of treatment, prevention or alleviation of obesity or an obesity associated disease of a living animal body, including a human, which method comprises the step of administering to such a living animal body in need thereof, a therapeutically effective amount of a compound of Formula I, any of its stereoisomers or any mixture of its stereoisomers, or a pharmaceutically acceptable salt thereof; and an anti-obesity compound or a pharmaceutically acceptable salt thereof.

Other objects of the invention will be apparent to the person skilled in the art from the following detailed description and examples.

### DETAILED DISCLOSURE OF THE INVENTION

In its first aspect the invention provides a pharmaceutical composition comprising a therapeutically effective amount of

(i) a compound of Formula I

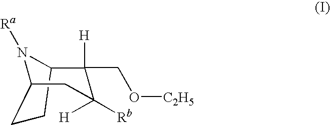

wherein

R^{a} represents hydrogen or alkyl;

R^{b} represents a dihalophenyl group;

any of its stereoisomers or any mixture of its stereoisomers, or a pharmaceutically acceptable salt thereof; and

(ii) an anti-obesity compound;

or a pharmaceutically acceptable salt thereof;

together with one or more adjuvants, excipients, carriers and/or diluents.

### Compounds of Formula I

The compounds of Formula I for use according to the invention are monoamine neurotransmitter re-uptake inhibitors, and are described in WO 97/30997 (NeuroSearch A/S). The compounds may be prepared by conventional methods for chemical synthesis, e.g. those described in WO 97/30997 and WO 2005/073228.

In one embodiment of the compound of Formula I, R^{a} represents hydrogen or methyl. In a special embodiment, R^{a} represents hydrogen. In a further embodiment, R^{a} represents methyl.

In a further embodiment of the compounds of Formula I, R^{b} represents dichlorophenyl. In a special embodiment, R^{b} represents 3,4-dichlorophenyl.

In a still further embodiment, the compound of Formula I is
tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo[3.2.1]octane;

or a pharmaceutically acceptable salt thereof.

In a special embodiment, the compound of Formula I is tesofensine or a pharmaceutically acceptable salt thereof. In a further special embodiment, the compound of Formula I is the citrate salt of tesofensine.

### Definition of Substituents

In the context of this invention halo represents fluoro, chloro, bromo or iodo.

In the context of this invention an alkyl group means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

### Steric Isomers

It will be appreciated by those skilled in the art that the compounds of Formula I may exist in different stereoisomeric forms—including enantiomers, diastereomers and cis-trans-isomers.

The invention includes all such isomers and any mixtures thereof including racemic mixtures.

### Anti-Obesity Compounds

The anti-obesity drug for use according to the invention is a compound different from the compound of Formula I. Preferably, the anti-obesity drug is selected from the following groups of compounds, which groups of compounds are known in the art and may be commercially available under different brand names, or may be obtained as described in the literature.

### Cannabinoid CB1 Receptor Antagonists

In one embodiment, the anti-obesity compound for use according to the invention is a cannabinoid CB1 receptor antagonist.

Examples of cannabinoid CB1 receptor antagonists include rimonabant, surinabant, SLV-319 [CAS Registry Number 464213-10-3] and O-2093 [CAS Registry Number 439080-01-0].

In one embodiment, the anti-obesity compound is selected from the group consisting of rimonabant, surinabant, SLV-319, O-2093 and pharmaceutically acceptable salts thereof.

### Lipase Inhibitors

In a further embodiment, the anti-obesity compound for use according to the invention is a lipase inhibitor.

Examples of lipase inhibitors include orlistat and cetilistat.

In one embodiment, the anti-obesity compound is selected from the group consisting of orlistat, cetilistat and pharmaceutically acceptable salts thereof.

### Monoamine Reuptake Inhibitors

In a still further embodiment, the anti-obesity compound for use according to the invention is a monoamine reuptake inhibitor, such as an SSRI or an SNRI.

Examples of monoamine reuptake inhibitors include sibutramine, bupropion, citalopram, escitalopram, fluoxetine, paroxetine, sertraline, duloxetine, milnacipran, mirtazapine, venlafaxine and desvenlafaxine.

In one embodiment, the anti-obesity compound is selected from the group consisting of sibutramine, bupropion, citalopram, escitalopram, fluoxetine, paroxetine, sertraline, duloxetine, milnacipran, mirtazapine, venlafaxine, desvenlafaxine, and pharmaceutically acceptable salts thereof.

### Anticonvulsants

In a further embodiment, the anti-obesity compound for use according to the invention is an anticonvulsant.

Examples of anticonvulsants include topiramate and zonisamide.

In one embodiment, the anti-obesity compound is selected from the group consisting of topiramate, zonisamide and pharmaceutically acceptable salts thereof.

### Glucose Sensitizers

In a still further embodiment, the anti-obesity compound for use according to the invention is a glucose sensitizer.

One example of a glucose sensitizer is metformin.

In one embodiment, the anti-obesity compound is selected from the group consisting of metformin and pharmaceutically acceptable salts thereof.

### Incretin Mimetics

In a further embodiment, the anti-obesity compound for use according to the invention is an incretin mimetic.

One example of an incretin mimetic is exenatide.

In one embodiment, the anti-obesity compound is selected from the group consisting of exenatide and pharmaceutically acceptable salts thereof.

### Amylin Analogs

In a still further embodiment, the anti-obesity compound for use according to the invention is an amylin analog.

One example of an amylin analog is pramlintide.

In a special, the anti-obesity compound is selected from the group consisting of pramlintide and pharmaceutically acceptable salts thereof.

### GLP-1 Analogs

In a further embodiment, the anti-obesity compound for use according to the invention is a GLP-1 analog.

One example of a GLP-1 analog is liraglutide.

In a special, the anti-obesity compound is selected from the group consisting of liraglutide and pharmaceutically acceptable salts thereof.

### Y Receptor Peptides

In a still further embodiment, the anti-obesity compound for use according to the invention is a Y receptor peptide.

One example of a Y receptor peptide is obinepitide.

In a special embodiment, the anti-obesity compound is selected from the group consisting of obinepitide and pharmaceutically acceptable salts thereof.

### 5-HT2C Serotonin Receptor Agonists

In a further embodiment, the anti-obesity compound for use according to the invention is a 5-HT2C serotonin receptor agonist.

One example of a 5-HT2C serotonin receptor agonist is lorcaserin.

In one embodiment, the anti-obesity compound is selected from the group consisting of lorcaserin and pharmaceutically acceptable salts thereof.

### Opioid Receptor Antagonists

In a still further embodiment, the anti-obesity compound for use according to the invention is an opioid receptor antagonist.

One example of an opioid receptor antagonist is naltrexone.

In one embodiment, the anti-obesity compound is selected from the group consisting of naltrexone and pharmaceutically acceptable salts thereof.

### Appetite Suppressants

In a further embodiment, the anti-obesity compound for use according to the invention is an appetite suppressant.

One example of an appetite suppressant is phentermine.

In one embodiment, the anti-obesity compound is selected from the group consisting of phentermine and pharmaceutically acceptable salts thereof.

### Anorectics

In a still further embodiment, the anti-obesity compound for use according to the invention is an anorectic.

One example of an anorectic is phendimetrazine.

In one embodiment, the anti-obesity compound is selected from the group consisting of phendimetrazine and pharmaceutically acceptable salts thereof.

### Hormones

In a still further embodiment, the anti-obesity compound for use according to the invention is a hormone.

Examples of hormones include insulin and leptin.

In one embodiment, the anti-obesity compound is selected from the group consisting of leptin and insulin and pharmaceutically acceptable salts thereof.

### Combinations of Compounds of Formula I and Anti-Obesity Compounds

In a special embodiment of the invention, the compound of Formula I is tesofensine or a pharmaceutically acceptable salt thereof and the anti-obesity compound is selected from the group consisting of: rimonabant, surinabant, SLV-319, O-2093, orlistat, cetilistat, sibutramine, bupropion, citalopram, escitalopram, fluoxetine, paroxetine, sertraline, duloxetine, milnacipran, mirtazapine, venlafaxine, desvenlafaxine topiramate, zonisamide, metformin, exenatide, pramlintide, liraglutide, obinepitide, lorcaserin, naltrexone, phentermine, phendimetrazine, insulin, leptin, and pharmaceutically acceptable salts thereof.

In a further special embodiment, the compound of Formula I is tesofensine or a pharmaceutically acceptable salt thereof and the anti-obesity compound is rimonabant or a pharmaceutically acceptable salt thereof.

In a still further special embodiment, the compound of Formula I is tesofensine or a pharmaceutically acceptable salt thereof and the anti-obesity compound is surinabant or a pharmaceutically acceptable salt thereof.

In a further special embodiment, the compound of Formula I is tesofensine or a pharmaceutically acceptable salt thereof and the anti-obesity compound is SLV-319 or a pharmaceutically acceptable salt thereof.

In a still further special embodiment, the compound of Formula I is tesofensine or a pharmaceutically acceptable salt thereof and the anti-obesity compound is O-2093 or a pharmaceutically acceptable salt thereof.

In a further special embodiment, the compound of Formula I is tesofensine or a pharmaceutically acceptable salt thereof and the anti-obesity compound is orlistat or a pharmaceutically acceptable salt thereof.

In a still further special embodiment, the compound of Formula I is tesofensine or a pharmaceutically acceptable salt thereof and the anti-obesity compound is cetilistat or a pharmaceutically acceptable salt thereof.

In a further special embodiment, the compound of Formula I is tesofensine or a pharmaceutically acceptable salt thereof and the anti-obesity compound is sibutramine or a pharmaceutically acceptable salt thereof.

In a still further special embodiment, the compound of Formula I is tesofensine or a pharmaceutically acceptable salt thereof and the anti-obesity compound is bupropion or a pharmaceutically acceptable salt thereof.

In a further special embodiment, the compound of Formula I is tesofensine or a pharmaceutically acceptable salt thereof and the anti-obesity compound is citalopram or a pharmaceutically acceptable salt thereof.

In a still further special embodiment, the compound of Formula I is tesofensine or a pharmaceutically acceptable salt thereof and the anti-obesity compound is escitalopram or a pharmaceutically acceptable salt thereof.

In a further special embodiment, the compound of Formula I is tesofensine or a pharmaceutically acceptable salt thereof and the anti-obesity compound is fluoxetine or a pharmaceutically acceptable salt thereof.

In a still further special embodiment, the compound of Formula I is tesofensine or a pharmaceutically acceptable salt thereof and the anti-obesity compound is paroxetine or a pharmaceutically acceptable salt thereof.

In a further special embodiment, the compound of Formula I is tesofensine or a pharmaceutically acceptable salt thereof and the anti-obesity compound is sertraline or a pharmaceutically acceptable salt thereof.

In a still further special embodiment, the compound of Formula I is tesofensine or a pharmaceutically acceptable salt thereof and the anti-obesity compound is duloxetine or a pharmaceutically acceptable salt thereof.

In a further special embodiment, the compound of Formula I is tesofensine or a pharmaceutically acceptable salt thereof and the anti-obesity compound is milnacipran or a pharmaceutically acceptable salt thereof.

In a still further special embodiment, the compound of Formula I is tesofensine or a pharmaceutically acceptable salt thereof and the anti-obesity compound is mirtazapine or a pharmaceutically acceptable salt thereof.

In a further special embodiment, the compound of Formula I is tesofensine or a pharmaceutically acceptable salt thereof and the anti-obesity compound is venlafaxine or a pharmaceutically acceptable salt thereof.

In a still further special embodiment, the compound of Formula I is tesofensine or a pharmaceutically acceptable salt thereof and the anti-obesity compound is desvenlafaxine or a pharmaceutically acceptable salt thereof.

In a further special embodiment, the compound of Formula I is tesofensine or a pharmaceutically acceptable salt thereof and the anti-obesity compound is topiramate or a pharmaceutically acceptable salt thereof.

In a still further special embodiment, the compound of Formula I is tesofensine or a pharmaceutically acceptable salt thereof and the anti-obesity compound is zonisamide or a pharmaceutically acceptable salt thereof.

In a further special embodiment, the compound of Formula I is tesofensine or a pharmaceutically acceptable salt thereof and the anti-obesity compound is metformin or a pharmaceutically acceptable salt thereof.

In a still further special embodiment, the compound of Formula I is tesofensine or a pharmaceutically acceptable salt thereof and the anti-obesity compound is exenatide or a pharmaceutically acceptable salt thereof.

In a further special embodiment, the compound of Formula I is tesofensine or a pharmaceutically acceptable salt thereof and the anti-obesity compound is pramlintide or a pharmaceutically acceptable salt thereof.

In a still further special embodiment, the compound of Formula I is tesofensine or a pharmaceutically acceptable salt thereof and the anti-obesity compound is liraglutide or a pharmaceutically acceptable salt thereof.

In a further special embodiment, the compound of Formula I is tesofensine or a pharmaceutically acceptable salt thereof and the anti-obesity compound is obinepitide or a pharmaceutically acceptable salt thereof.

In a still further special embodiment, the compound of Formula I is tesofensine or a pharmaceutically acceptable salt thereof and the anti-obesity compound is lorcaserin or a pharmaceutically acceptable salt thereof.

In a further special embodiment, the compound of Formula I is tesofensine or a pharmaceutically acceptable salt thereof and the anti-obesity compound is naltrexone or a pharmaceutically acceptable salt thereof.

In a still further special embodiment, the compound of Formula I is tesofensine or a pharmaceutically acceptable salt thereof and the anti-obesity compound is phentermine or a pharmaceutically acceptable salt thereof.

In a further special embodiment, the compound of Formula I is tesofensine or a pharmaceutically acceptable salt thereof and the anti-obesity compound is phendimetrazine or a pharmaceutically acceptable salt thereof.

In a still further special embodiment, the compound of Formula I is tesofensine or a pharmaceutically acceptable salt thereof and the anti-obesity compound is insulin or a pharmaceutically acceptable salt thereof.

In a further special embodiment, the compound of Formula I is tesofensine or a pharmaceutically acceptable salt thereof and the anti-obesity compound is leptin or a pharmaceutically acceptable salt thereof.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound for use according to the invention include examples of suitable prodrugs of the substances for use according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compounds for use according to the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Biological Activity

The pharmaceutical compositions for use according to the invention are contemplated particularly useful for combating obesity or an obesity associated disease.

In one embodiment obesity or an obesity associated disease is a disorder or condition selected from the group consisting of obesity, over-eating disorders, bulimia nervosa, binge eating disorder (BED), compulsive over-eating, impaired appetite regulation, metabolic syndrome, type 2 diabetes, dyslipidemia, atherosclerosis.

The pharmaceutical compositions for use according to the invention are also contemplated useful for obesity management, obtaining weight loss, weight maintenance, and weight maintenance used in conjunction with a reduced-calorie diet.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In a preferred embodiment, the invention provides pharmaceutical compositions comprising the active compounds or pharmaceutically acceptable salts or derivatives thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

### Dosages

The actual dosage of each of the active ingredients depends on the nature and severity of the disease being treated, the exact mode of administration, form of administration and is within the discretion of the physician, and may be varied by titration of the dosage to the particular circumstances of this invention to produce the desired therapeutic effect. However, the below dosages for the compound of Formula I and the anti-obesity compound are considered suitable.

The dosage of the compound of Formula I is determined as the API (Active Pharmaceutical Ingredient), i.e. calculated as the free base.

A daily dosage in the range of about 0.1-2 mg API daily, preferably of about 0.25-1 mg API daily, especially 0.25, 0.5 or 1.0 mg API daily, is suitable for therapeutic treatments. The daily dosage of the compound of Formula I may be administered in one or several doses, such as two, per day. In one embodiment, the daily dosage is administered in one dose.

The daily dosage of the anti-obesity compound is presently contemplated to be in the range of about 0.1-500 mg of active ingredient depending on the actual compound. More specific dosage intervals may be in the range of about 0.1-2 mg, about 1-10 mg, about 10-50 mg, about 25-100 mg, about 50-200 mg and about 100-500 mg daily. The daily dosage of the anti-obesity compound may be administered in one or several doses, such as two, per day. In one embodiment, the daily dosage is administered in one dose.

### Pharmaceutical Kits of Parts

According to the invention there is also provided a kit of parts comprising at least two separate unit dosage forms (A) and (B):

(A) a compound of Formula I

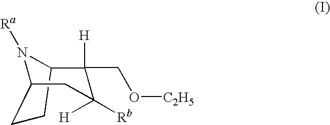

wherein

R^{a} represents hydrogen or alkyl;

R^{b} represents a dihalophenyl group;

any of its stereoisomers or any mixture of its stereoisomers, or a pharmaceutically acceptable salt thereof; and

(B) an anti-obesity compound;

or a pharmaceutically acceptable salt thereof; and optionally

(C) instructions for the simultaneous, sequential or separate administration of the compound of (A) and the anti-obesity compound of (B) to a patient in need thereof.

The compound of Formula I for use according to the invention and the anti-obesity compound for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the compound of Formula I for use according to the invention and the anti-obesity compound for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the compound of Formula I and the anti-obesity compound are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Therapy

In another aspect the invention provides methods of treatment, prevention or alleviation of obesity or an obesity associated disease of a living animal body, including a human, which method comprises the step of administering to such a living animal body in need thereof, a therapeutically effective amount of a combination of a compound of Formula I, any of its stereoisomers or any mixture of its stereoisomers, or a pharmaceutically acceptable salt thereof and an anti-obesity compound or a pharmaceutically acceptable salt thereof.

The preferred indications contemplated according to the invention are those stated above.

When administered in combination with further compounds known in the art for treatment of the diseases, the dose regimen may be reduced.

1. A pharmaceutical composition comprising a therapeutically effective amount of
(i) a compound of Formula I 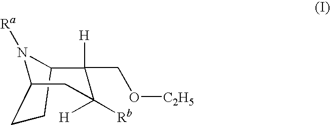
wherein
R^{a} represents hydrogen or alkyl;
R^{b} represents a dihalophenyl group;
any of its stereoisomers or any mixture of its stereoisomers, or a pharmaceutically acceptable salt thereof; and
(ii) an anti-obesity compound;
or a pharmaceutically acceptable salt thereof;
together with one or more adjuvants, excipients, carriers and/or diluents.

2. The pharmaceutical composition of claim 1 wherein R^{a} represents hydrogen or methyl.

3. The pharmaceutical composition of claim 1 or 2, wherein R^{b} represents 3,4-dichlorophenyl.

4. The pharmaceutical composition of claim 1 wherein the compound of Formula I is
tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo[3.2.1]octane;
or a pharmaceutically acceptable salt thereof.

5. The pharmaceutical composition of claim 1, wherein the anti-obesity compound is a cannabinoid CB1 receptor antagonist, a lipase inhibitor, a monoamine reuptake inhibitor, an anticonvulsant, a glucose sensitizer, a incretin mimetic, an amylin analog, a GLP-1 analog, a Y receptor peptide, a 5-HT2C serotonin receptor agonist, an opioid receptor antagonist, an appetite suppressant, an anorectic, a hormone; or a pharmaceutically acceptable salt thereof.

6. The pharmaceutical composition of claim 5, wherein the anti-obesity compound is selected from the group consisting of: rimonabant, surinabant, SLV-319, O-2093, orlistat, cetilistat, sibutramine, bupropion, citalopram, escitalopram, fluoxetine, paroxetine, sertraline, duloxetine, milnacipran, mirtazapine, venlafaxine, desvenlafaxine, topiramate, zonisamide, metformin, exenatide, pramlintide, liraglutide, obinepitide, lorcaserin, naltrexone, phentermine, phendimetrazine, insulin, leptin, and pharmaceutically acceptable salts thereof

7-12. (canceled)

13. A method of treatment, prevention or alleviation of obesity or an obesity associated disease of a living animal body, including a human, which method comprises the step of administering to such a living animal body in need thereof, a therapeutically effective amount of a combination of
(i) a compound of Formula I 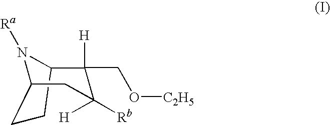
wherein
R^{a} represents hydrogen or alkyl;
R^{b} represents a dihalophenyl group;
any of its stereoisomers or any mixture of its stereoisomers, or a pharmaceutically acceptable salt thereof; and
(ii) an anti-obesity compound;
or a pharmaceutically acceptable salt thereof.

14. The method according to claim 13, wherein obesity or an obesity associated disease is a disorder or condition selected from the group consisting of obesity, over-eating disorders, bulimia nervosa, binge eating disorder (BED), compulsive over-eating, impaired appetite regulation, metabolic syndrome, type 2 diabetes, dyslipidemia, atherosclerosis.

15. A kit of parts comprising at least two separate unit dosage forms (A) and (B):
(A) a compound of Formula I 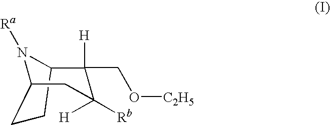
wherein
R^{a} represents hydrogen or alkyl;
R^{b} represents a dihalophenyl group;
any of its stereoisomers or any mixture of its stereoisomers, or a pharmaceutically acceptable salt thereof; and
(B) an anti-obesity compound;
or a pharmaceutically acceptable salt thereof; and optionally
(C) instructions for the simultaneous, sequential or separate administration of the compound of (A) and the anti-obesity compound of (B) to a patient in need thereof.

16. A combination of
(i) a compound of Formula I 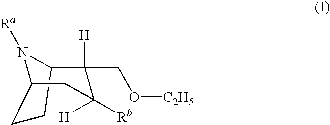
wherein
R^{a} represents hydrogen or alkyl;
R^{b} represents a dihalophenyl group;
any of its stereoisomers or any mixture of its stereoisomers, or a pharmaceutically acceptable salt thereof; and
(ii) an anti-obesity compound;
or a pharmaceutically acceptable salt thereof;
for use as a medicament.

17. A combination of
(i) a compound of Formula I 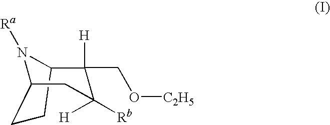
wherein
R^{a} represents hydrogen or alkyl;
R^{b} represents a dihalophenyl group;
any of its stereoisomers or any mixture of its stereoisomers, or a pharmaceutically acceptable salt thereof; and
(ii) an anti-obesity compound;
or a pharmaceutically acceptable salt thereof;
for use in the treatment, prevention or alleviation of obesity or an obesity associated disease of a living animal body, including a human.
